(19) United States
(12) Patent Application Publication
(10) Pub. No.: US 2025/0266167 A1
(43) Pub. Date: Aug. 21, 2025

(54) MACHINE LEARNING TECHNIQUES TO ASSIST DIAGNOSIS OF EAR DISEASES

(71) Applicant: REMMIE, INC., Wilmington, DE (US)

(72) Inventors: Jane Yuqian ZHANG, Bothell, WA (US); Zhan WANG, Newcastle, WA (US); Emma Oo, Duarte, CA (US)

(73) Assignee: REMMIE, INC., Wilmington, DE (US)

(21) Appl. No.: 19/199,378

(22) Filed: May 5, 2025

Related U.S. Application Data
(63) Continuation of application No. 17/508,517, filed on Oct. 22, 2021.
(63) Continuation of application No. PCT/US2024/026259, filed on Apr. 25, 2024.
(60) Provisional application No. 63/104,932, filed on Oct. 23, 2020.
(60) Provisional application No. 63/461,815, filed on Apr. 25, 2023.

(51) Int. Cl. G16H 50/20; G16H 30/20

(52) U.S. Cl. CPC G16H 50/20; G16H 30/20

(57) ABSTRACT

Various aspects of methods, systems, and use cases may be used to generate an ear disease state prediction to assist diagnosis of an ear disease. A method may include receiving an image an ear, predicting an image-based confidence level of a disease state in the ear by using the image as in input to a machine learning trained model. The method may include receiving text, for example corresponding to a symptom of the patient. The method may include predicting a symptom-based confidence level of the disease state in the ear by using the text as in input to a trained classifier. The method may include using the results of the image-based confidence level and the symptom-based confidence level to determine an overall confidence level of presence of an ear infection in the ear of the patient.

### CLAIM OF PRIORITY

This application is a continuation-in-part of U.S. application Ser. No. 17/508,517, filed on Oct. 22, 2021, and International Application No. PCT/US2024/026259, filed on Apr. 25, 2024. U.S. application Ser. No. 17/508,517 claims the benefit of priority to U.S. Provisional Application No. 63/104,932 filed Oct. 23, 2020. International Application No. PCT/US2024/026259 claims the benefit of priority to U.S. Provisional Application No. 63/461,815, filed on Apr. 25, 2023. The disclosures of all of the foregoing applications are hereby incorporated herein by reference in their entireties.

### BACKGROUND

Acute Otitis Media (AOM, or ear infection) is the most common reason for a sick child visit in the US as well as low to mid income countries. Ear infections account for the most common reason for antibiotics usage for children under 6 years, particularly in the 24-month to 3 age group. It is also the second most important cause of hearing loss, impacting 1.4 billion in 2017 and ranked fifth highest disease burden globally.

During a physician's visit, the standard practice for diagnosing an AOM requires inserting an otoscope with a disposable speculum in the external ear along the ear canal to visualize the tympanic membrane (eardrum). A healthy eardrum appears clear and pinkish-gray, whereas an infected one will appear red and swollen due to fluid buildup behind the membrane. Access to otolaryngology, pediatric, or primary specialist is severely limited in low resource settings, leaving AOM undiagnosed or misdiagnosed. The primary unmet needs with an ear infection are the lack of means to track disease progression, which could lead to delayed diagnosis at onset or ineffective treatment.

During a physician's visit, an otoscope with a disposable speculum is inserted in the external ear along the ear canal to visualize the tympanic membrane (eardrum). A healthy eardrum appears clear and pinkish-gray, whereas an infected one will appear red and swollen due to fluid buildup behind the membrane. However, these features are not immediately distinguishable especially when there is limited time to view the eardrum especially of a squirmy child using a traditional otoscope.

Telemedicine provides a viable means for in-home visits to a provider with no wait time and closed-loop treatment guidance or prescription. An ear infection is an ideal candidate for real-time telemedicine visits, but due to the lack of means to visualize inside the ear, telemedicine provider cannot accurately diagnose an ear infection. As a result, telemedicine was found to lead to over-prescription of antibiotics or “new utilization” of clinical resources which would otherwise not occur compared to in-person visits.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates a platform for ear nose and throat disease state diagnostic support in accordance with at least one example of this disclosure.

FIG. 1B illustrates an example of a system for improving dataset quality via an object detection model.

FIG. 2 illustrates a system for training and implementing the multi-model with image and text classification models for predicting outcomes related to ear nose and throat disease state in accordance with at least one example of this disclosure.

FIG. 3A illustrates examples of a healthy eardrum and an infected eardrum in accordance with at least one example of this disclosure.

FIG. 3B illustrates an example of data augmentation to generate training data in accordance with at least one example of this disclosure.

FIG. 3C illustrates an example of image segmentation in accordance with at least one example of this disclosure.

FIGS. 4A-4B illustrate results of image and text classification predictions in accordance with at least one example of this disclosure.

FIG. 5 illustrates a flowchart showing a technique for generating an ear disease state prediction to assist diagnosis of an ear disease in accordance with at least one example of this disclosure.

FIG. 6 illustrates a block diagram of an example machine upon which any one or more of the techniques discussed herein may perform in accordance with at least one example of this disclosure.

FIG. 7 illustrates an example augmented image of the eardrum with Malleus handle displayed on a user interface.

FIG. 8A illustrates an example of an otoscope with one or more additional components.

FIG. 8B illustrates an example of an architecture for an object detection model.

FIG. 9A illustrates an example of a suitable image frame, including at least one target feature (either Eardrum and/or Malleus handle), for object detection or classification of Otitis Media.

FIG. 9B illustrates an example of an unsuitable image frame, not including at least one target feature, for object detection or classification of Otitis Media.

FIG. 10A illustrates an example of an image frame before augmentation.

FIG. 10B illustrates an example of the image frame of FIG. 5A after augmentation.

FIG. 11A illustrates an example of an image frame.

FIG. 11B illustrates the image frame of FIG. 11A, following a cropping operation.

FIG. 11C illustrates the image frame of FIG. 11B, following a random rotation operation.

FIG. 11D illustrates the image frame of FIG. 11C, following a scaling operation and a cropping operation.

FIG. 11E illustrates the image frame of FIG. 11D, following a flipping transformation operation.

FIG. 11F illustrates the image of frame of FIG. 11E, following a normalization operation.

FIG. 12 illustrates machine learning engine for training and execution related to object detection.

FIG. 13 illustrates a flowchart showing a technique for training the model and identifying an anatomical object of a middle ear in an image.

FIG. 14 illustrates a flowchart showing a technique for training the model and identifying the presence of Otitis Media (OM), e.g., at or near a middle ear in an image frame.

### DETAILED DESCRIPTION

A system and method for early and remote diagnosis of ear disease is disclosed. An images of a patient's inner ear may be taken with an otoscope and transmitted to a cloud-based database. A machine learning-based algorithm is used to classify images for presence or absence of diseases such as AOM, and other diagnosis. The results of the classification and diagnosis may be sent to third parties such as physicians, healthcare providers to be integrated in patient care decisions.

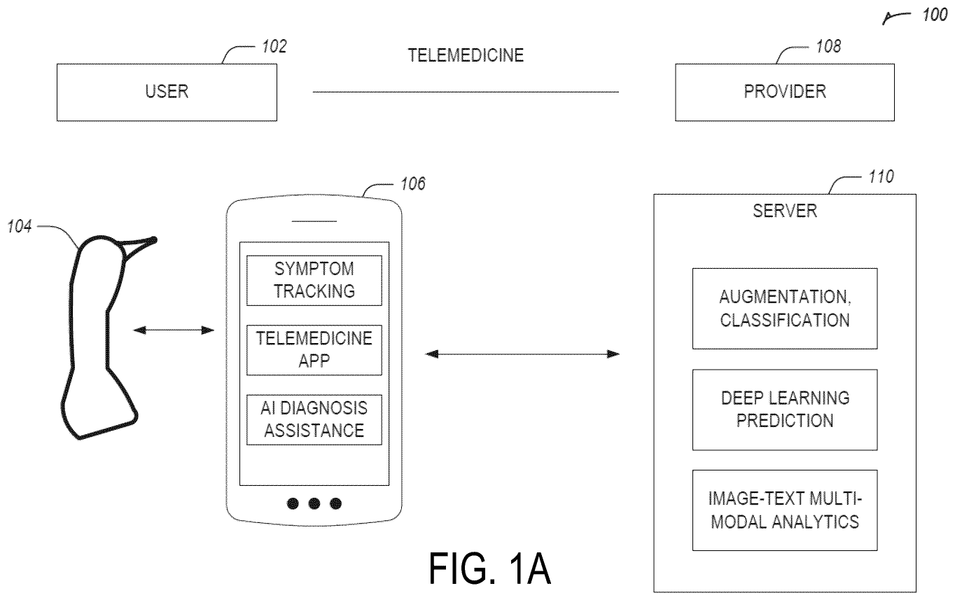

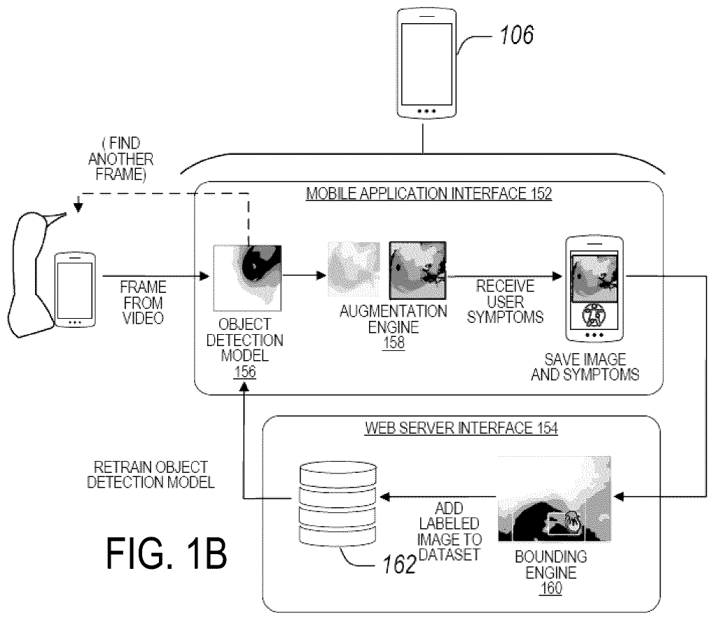

An otitis media is most commonly diagnosed using an otoscope (FIG. 1), essentially a light source with a magnifying eyepiece for visualization of the ear canal and eardrum with the human eye. The key features of these currently commercially available products are summarized in Table 1. These otoscopes lack communication functions requisite for the current invention but can be incorporated after the communication functions are fulfilled by complementing devices.

In one embodiment, an otoscope is disclosed that is configured to be used together with a host device, such as a smart phone or other handheld mobile devices. The host device can be used to capture images. The images can be uploaded to cloud-based database. The images can be shared through an app in the host device. The uploaded images are labelled with respective clinical diagnosis.

In one embodiment, the uploaded images can be used as data source to train the algorithm. At least 500 “normal”, 300 AOM images, and additional images with “other” ailments (O/W, OME, and CSOM) are collected for training purposes. The images are de-identified and securely stored for subsequent analysis.

In normal operation of an otoscope, the eardrum may be visualized in varying regions of the field of view. Translation of images will make the algorithm location invariant.

FIG. 1A illustrates a platform 100 for ear nose and throat disease state diagnostic support in accordance with at least one example of this disclosure. The platform 100 includes a user ecosystem 102 and a provider ecosystem 108. The two ecosystems 102 and 108 may perform various functions, with some overlap and some unique to the ecosystem. In some examples, the user ecosystem 102 and the provider ecosystem 108 are remote from each other (e.g., a patient may be at home using the user ecosystem 102, while a doctor operates the provider ecosystem 108 from an office), and in other examples the ecosystems 102 and 108 may be local to each other, such as when a patient visits a doctor's office. The devices of the user ecosystem 102 and the provider ecosystem 108 may communicate (e.g., via a network, wirelessly, etc.) with each other and with devices within each ecosystem.

In an example, the user ecosystem 102 includes an otoscope 104 and a user device 106 (e.g., a mobile device such as a phone or a tablet, a computer such as a laptop or a desktop, a wearable, or the like). The otoscope 104 may be communicatively coupled to the user device 106 (e.g., configured to send data such as an image over a wired or wireless connection, such as Bluetooth, Wi-Fi, Wi-Fi direct, near field communication (NFC), or the like). In some examples, functionality of the otoscope 104 may be controlled by the user device 106. For example, the user device 106 may trigger a capture of an image or video at the otoscope 104. The triggering may be caused by a user selection on a user interface on the user device 106, caused automatically (e.g., via a detection of an object within a camera view of the otoscope 104, such as an ear drum), or via remote action (e.g., by a device of the provider ecosystem 108). When the trigger is via a remote action, the remote action may include a provider selection on a user interface of a device of the provider ecosystem 108 indicating that the camera view of the otoscope 104 is acceptable (e.g., a capture will include an image of an ear drum or other anatomical feature of a patient).

The otoscope 104 may be used to capture an image of an ear drum or inner ear portion of a patient. When the image is captured, it may be sent to the user device 106, which may in turn send the image a device of the provider ecosystem 108, such as a server 110. In another example, the image may be sent directly from the otoscope 104 to the server 110. The user device 106 may receive an input including text on a user interface by a patient in some examples, such as user entered text, a selection of a menu item, or the like. The user input may include a text representation of a symptom (e.g., fever, nausea, sore throat, etc.). The user input may be sent from the user device 106 to the server 110.

The user device 106 may be used to track symptoms, place or receive secure calls or send or receive secure messages to a provider, or perform AI diagnostic assistance. The server 110 may be used to place or receive secure calls or send or receive secure messages with the user device 106. The server 110 may perform augmentation classification to train a model (e.g., the AI diagnosis assistant), use a model to perform a deep learning prediction, or perform image-text multi-modal analytics. In some examples, the server 110 or the user device 106 may output a prediction for diagnosis assistance, such as a likelihood of a patient having an ear infection. The prediction may be based on images captured by the otoscope 104 input to a deep learning model. The prediction may be based on text received via user input at the user device 106 (or over a phone call with a provider, entered by the provider) input to a text classifier. The prediction may be based on an output of both the deep learning model and the text classifier (e.g., a combination, such as by multiplying likelihoods together, taking an average likelihood, using one of the results as a threshold, etc.).

FIG. 1B illustrates an example of a system for improving dataset quality via an object detection model. The system 150 can include a mobile application interface 152 (e.g., accessible via the user device 106), and a web server interface 154 for accessing the server 110. The system 150 can facilitate processing/filtering, color correction, user interaction with a patient such as prompting the user to capture the correct location of the eardrum images with bounding boxes, bounding/labeling of images, and training/retraining of a machine learning model to identify an anatomical object.

In an example, the mobile application interface 152 can receive a frame from a live video feed of the otoscope 104. The object detection model 156 can facilitate near-real-time responses (e.g., less than about 30 seconds or less than about 5 seconds) to a patient user operating the otoscope 104. For example, the object detection model 156 can be deployed in the mobile application interface 152, such as deployed offline or without a need for communication to another cloud-based service. In an example, the object detection model 156 can receive images from the otoscope at a frequency greater than 1 image per second and process the images locally, on the user device 106. When the object detection model 156 detects at least one of an eardrum or malleus handle a relatively center (e.g., within about a middle third) of the field of view, the mobile application interface 152 can prompt the user to capture an image via the otoscope 104 for use in diagnosis, transmission to a medical professional, or uploading to the web server interface 154. In an example, the object detection model 156 can receive images from the live video feed, the captured image, or both via the otoscope 104 and determine whether an individual image is suitable for object identification or medical classification.

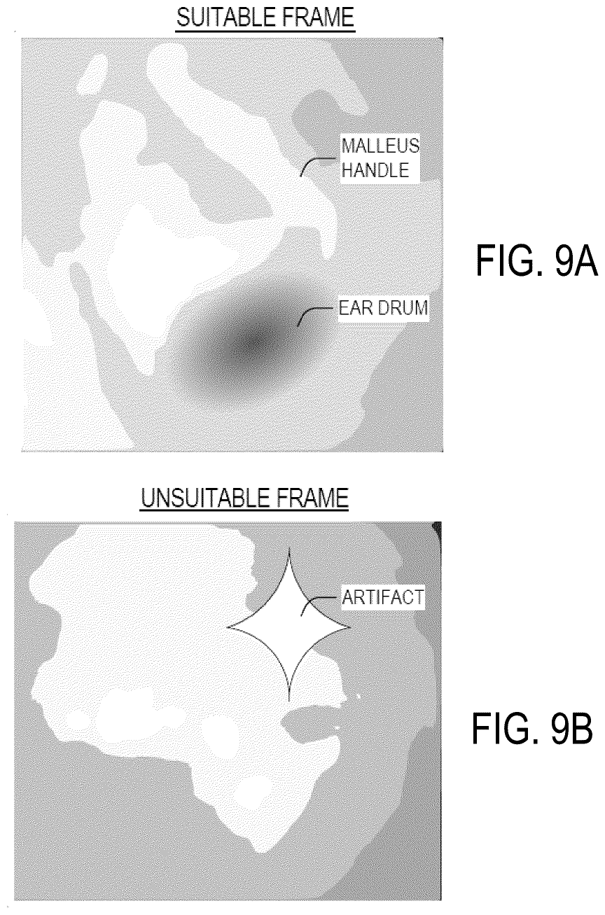

FIG. 9A and FIG. 9B depict examples of suitable and unsuitable frames, respectively. In an example, the mobile application interface 152 of FIG. 1B can facilitate that received images exhibit (e.g., automatically determined by the object detection model 156 or manually by a technician) characteristics that satisfy at least one of the following parameters: (1) a feature resembling a malleus handle of a patient ear is visible, (2) a feature resembling an eardrum of the patient ear clearly visible, or (3) no significant artifacts such as glare, blur, or occlusion are present in the captured image. In an example, the mobile application interface 152 can facilitate that the captured image satisfies each of parameters (1), (2), and (3) (such as the exemplary “suitable” frame depicted in FIG. 9A. In an example, the mobile application interface 152 can filter out or discard images not exhibiting characteristics that satisfy a specified, requisite set of parameters. Where features resembling a malleus handle or an eardrum are not present, or where an artifact such as glare, blur, or occlusion is present, the mobile application interface 152 can prompt the user (e.g., a patient user) to recapture an image with the otoscope 104. For example, the mobile application interface 152 can provide instructions or additional information to guide the user in recapturing an image. Alternatively or additionally, the object detection model 156 can be hosted via the server 110 and can be accessed remotely via the mobile application interface 152.

Once the mobile application interface 152 has received a captured image, the mobile application interface 152 can facilitate processing/filtering of the image via an augmentation engine 158. For example, the augmentation engine 158 can modify at least one visual characteristic of the image to aid in identification of a target object and improve a training set. In an example, the augmentation engine 158 can include at least one of a color correction block, a spatial distortion block, or a labeling block. The color correction block can facilitate at least one of contrast correction, channel-wise (e.g., RGB) color normalization, histogram equalization, or contrast stretching.

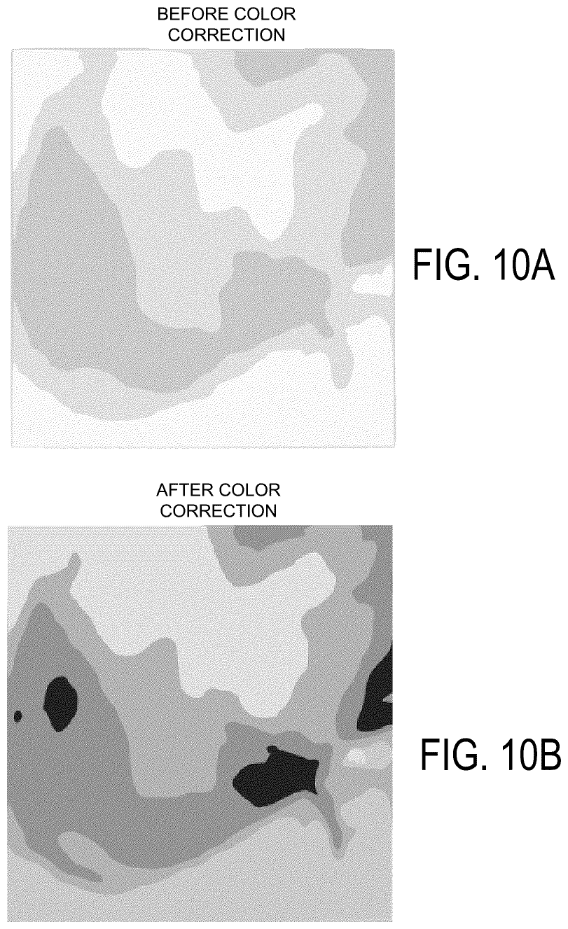

FIG. 10A and FIG. 10B depict an unaugmented image frame and an augmented image frame, respectively. For example, the image frame in FIG. 10A can be determined to be suitable (e.g., with respect to at least one of the above parameters (1), (2), or (3)), but lacking in a visual quality or characteristic. For example, the unaugmented image frame can exhibit noise, lack a desired contrast level, lack a desired brightness level, or lack sharpness. The augmentation engine 158 of FIG. 1B can facilitate color correction, e.g., to augment the image frame in FIG. 10A and to produce the augmented image frame in FIG. 10B. For example, the color correction block can facilitate a shading correction to remove artifacts caused by a camera lens or inconsistent illumination. Furthermore, the color correction block can facilitate spectral color correction to adjust colors or saturation. Alternatively or additionally, the color correction block can facilitate contrast correction via interpolation between a darkest pixel of the image frame and a lightest pixel of the image frame. Here, the interpolation can be conducted as a visible-light transform or may be run pixel by pixel to adjust the contrast level. In an example, the color correction block can approximate a distribution of a normal distribution and adjust the contrast level accordingly said distribution such that undesirable pixels in the image frame can be brought within a desired contrast level. In an example, the color correction block can facilitate channel-wise (such as green-channel biased) normalization to adjust channel levels. In another example, the color correction block can carry out a multi-pass histogram equalization using grayscale therapy values to heighten features in a captured image and amplify the contrast of the captured image. For example, the augmentation engine 158 can facilitate the use of convolutional neural networks with either artificial or natural images to generate synthetic and enhanced versions of such images. In an example, the color correction block can facilitate color normalization of an entire dataset (e.g., dataset 162) via RGB mean subtraction, and the normalized dataset can be divided by the standard deviation values of the images in the dataset 162.

The spatial distortion block can facilitate at least one of lens distortion correction, background subtraction, foreground clustering, a visibility score, or an object tracking algorithm with the use of deformation filters and model filters. The background subtraction block can facilitate removal or manipulation of the background of a captured image, e.g., via hue, saturation, or brightness filtering.

The spatial distortion block can facilitate at least one of rotation, scaling, translation, affine methods, perspective distortion, scale-based jittering, elastic deformation, or warp-based transformations.

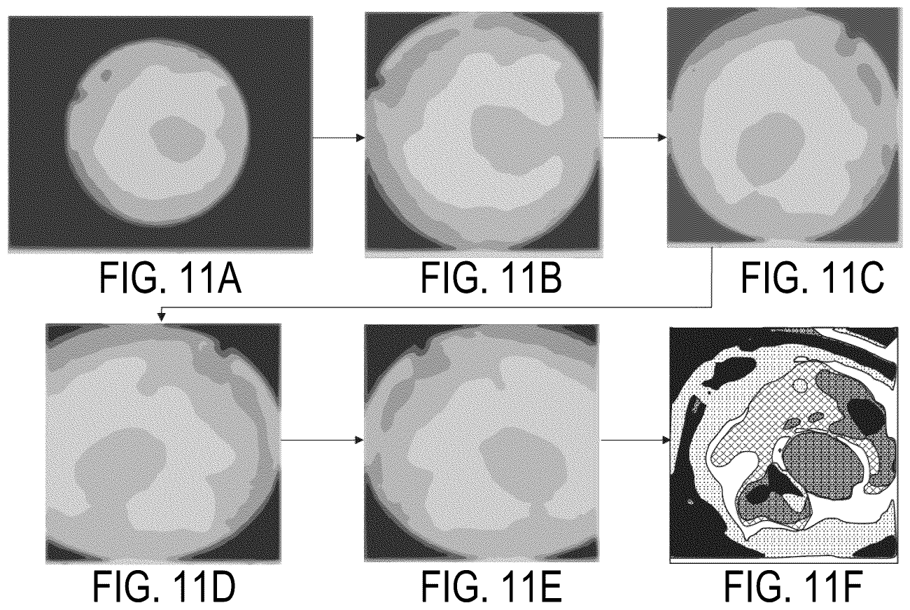

FIG. 11A, FIG. 11B, FIG. 11C, FIG. 11D, FIG. 11E, and FIG. 11F depict a progression of spatial distortion transformations of an image frame. For example, at FIG. 11A an image frame can be received by the spatial distortion block. The image frame can be otherwise unaugmented before spatial distortion or can have been altered or enhanced via the color correction block or the labeling block before being received by the spatial distortion block. As illustrated in FIG. 11B, the image frame of FIG. 11A, can be cropped for the purpose of zooming in on a subject object while discarding a surrounding object or the background. As illustrated in FIG. 11C, the image frame of FIG. 11B can be translated via a random rotation operation, for the purpose of reducing observational bias or user-induced irregularities in the image capture via the otoscope. As illustrated in FIG. 11D the image frame of FIG. can be scaled and optionally further cropped. For example, the image can be scaled to a resolution less than 250×250 pixels (e.g., a resolution of about 224×224 pixels) such that the image is suitable for downstream receipt via a model (e.g., the object detection model 156 of FIG. 1B or a classification model). As illustrated in FIG. 11E, the image frame of FIG. 11D can be flipped, mirrored, or reflected via a transformation operation. Flipping of the image frame can be performed to further reduce observational bias or user-induced irregularities. As illustrated in FIG. 11F the image of frame of FIG. 11E can be normalized via a color normalization operation. For example, the color normalization operation can be informed by histogram data or intensity data of the image frame to color normalize each of the red, green, and blue channels of the image. Further normalization of the color channels can be performed, e.g., via green channel-based normalization or contrast stretching. In an example, the spatial distortion block can use the deformation filters and the model filters to perform rotating, scaling and mixing operations on visual source material (e.g., for image frames). Such a progression of successive augmentation steps (as in FIG. 11A-FIG. 11F) can improve a training rate of a downstream of robust machine learning model such as by facilitating motif identifications or providing additional examples that can be used in training.

Returning to FIG. 1B, the labeling block of the augmentation engine 158 can facilitate creating a new label for the processed image of corresponding object using spatial information relating to the original image, with potential inclusion of at least one keypoint estimation layer via landmarks in the image (e.g., vertex points or feature corners for an individual object-such as vertex points or the feature corners of an eardrum). In an example, each label corresponds to a specific object present in the training set and can specify at least one of an image class (e.g., eyeglasses, earrings, blemish or blur, misfit), a quality tag such as “suitable” or “unsuitable”, and a segment or mask of the specified object within the original captured image. Alternatively, the mobile application interface 152 and the augmentation engine 158 can not facilitate a labeling function and reserve labeling, bounding, or other classification steps for the web server interface 154 or the server 110.

In an example, the mobile application interface can facilitate receiving information indicating a user symptom. For example, the information indicating a user symptom can be obtained from a choice received form by the user, manually input from the user (e.g., a selection of pain severity, discomfort level, or input of a narrative symptom), via selection of one or more highlighted regions in the image or over the car-face region of interest itself. The mobile application interface 152 can facilitate chatbot virtual assistant dialogue or human-based dialog process for obtaining and recording a user symptom. The information indicating a user symptom can be compared to the image, such as via a non-parametric classifier, to produce a usability classification. The usability classification can be used to compare the information indicating a symptom between different received images for improved detection of correlations between image-symptom pairs.

Following augmentation via the augmentation engine 158 of the mobile application interface 152, the processed/filtered image frame (and any corresponding symptoms or diagnostic information corresponding therewith) can be saved locally or uploaded to the cloud-based server 110, such as for further processing via the web server interface 154. In an example, the web server interface 154 includes a bounding engine 160 by an object detection model configured to identify, label, or document locations or regions of interest within the captured image requiring further polling for pathology. For example, the bounding engine 160 can apply an image segmentation strategy to label regions of the captured image as containing tissue or other anatomical material. The localization or bounding of a region of interest can consider a coloration, a curvature, or a profile found in the captured image. Labeling the regions of interest can aid a physician or a user during later diagnosis sessions.

Following bounding or labeling via the bounding engine 160, the image can be added to a dataset 162. In an example, the dataset 162 can include unlabelled and unclassified images. The dataset 162 can also include labeled or segmented, anonymized images collected via a medical institution. For example, each data point in the dataset 162 can correspond with an image of a patient's eardrum and their corresponding diagnosis via a medical professional, symptoms, or medical outcome. Table 1 shows a representation of a dataset, following binarization for classification of Otitis Media (OM).

TABLE 1

| | % of | | | |
|---|---|---|---|---|
| Binary | Binary | | Number of | % of |
| Condition | Classes | Condition | Samples | Dataset |
| Otitis Media | 38.43 | AOM | 247 | 8.21 |
| | | ASOM | 248 | 8.24 |
| | | CSOM | 246 | 8.18 |
| | | OME | 250 | 8.31 |
| Not Otitis Media | 61.57 | Otitis Externa | 249 | 8.28 |
| | | Abnormal | 59 | 1.96 |
| | | Pinna | | |
| | | Foreign Body | 249 | 8.28 |
| | | Fungal | 250 | 8.31 |
| | | Infection | | |
| | | Impacted | 250 | 8.31 |
| | | Wax | | |
| | | Inflammation | 223 | 7.41 |
| | | of Pinna | | |
| | | Normal Ears | 250 | 8.31 |
| | | Nose Throat | 58 | 1.93 |
| | | Disorders | | |
| No Visible | | Diminished | 180 | 5.98 |
| Characteristics | | Hearing | | |
| | | Tinnitus | 249 | 8.28 |
| Total (Binary) | | Otitis Media | 2579 | 85.7 |
| | | and | | |
| | | Not | | |
| | | Otitis | | |
| | | Media | | |
| Total | | | 3008 | 100 |

Table 2 shows an example of a representation of another binarized dataset for classification of Otitis Media. The representation of the dataset shown in the example of table 2 can be further idealized, such as to demonstrate a proof-of-concept in use of a machine learning model. For example, the dataset corresponding with table 2 can be used as a control in an experiment.

TABLE 2

| Binary | % of Binary | | Number of | % of |
|---|---|---|---|---|
| Condition | Classes | Condition | Photos | Dataset |
| Otitis Media | 40 | AOM | 15 | 7.14 |
| | | ASOM | 15 | 7.14 |
| | | CSOM | 15 | 7.14 |
| | | OME | 15 | 7.14 |
| Not Otitis Media | 60 | Otitis Externa | 15 | 7.14 |
| | | Abnormal | 15 | 7.14 |
| | | Pinna | | |
| | | Foreign Body | 15 | 7.14 |
| | | Fungal | 15 | 7.14 |
| | | Infection | | |
| | | Impacted Wax | 15 | 7.14 |
| | | Inflammation | 15 | 7.14 |
| | | of Pinna | | |
| | | Normal Ears | 15 | 7.14 |
| | | Nose Throat | 15 | 7.14 |
| | | Disorders | | |
| No Visible | | Diminished | 15 | 7.14 |
| Characteristics | | Hearing | | |
| | | Tinnitus | 15 | 7.14 |
| Total (Binary) | | Otitis Media | 180 | 85.7 |
| | | and | | |
| | | Not | | |
| | | Otitis | | |
| | | Media | | |
| Total | | | 210 | 100 |

In an example, the dataset 162, including new images having been received via the mobile application interface 152 (and, e.g., augmented via the augmentation engine 158) can be used to retrain the object detection model 156 or another machine learning model. In an example, such a model can be retrained to achieve a highly consistent classifier with a precision greater than about 85% and a recall greater than about 90%.

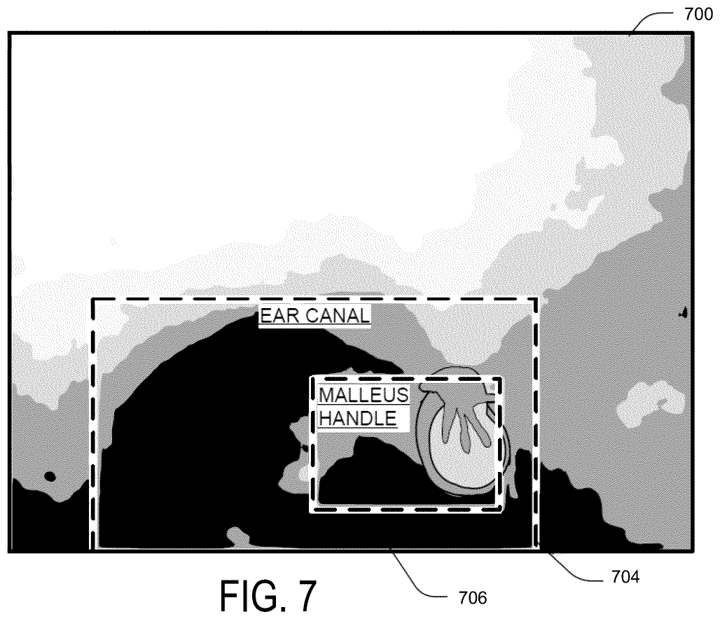

FIG. 7 illustrates an example augmented image 700 displayed on a user interface, in accordance with at least one example of this disclosure. The augmented image 700 may be displayed on a patient device (e.g., a mobile device) or a clinician device (e.g., on a computer of a physician). The augmented image 700 may include an identified feature (e.g., of an ear, nose, or throat), such as an ear canal 704 or a malleus handle 706. An identified feature may be identified using a trained machine learning model as described herein. The augmented image 700 may be displayed in real-time or near real-time (e.g., with a delay of less than a second) or saved for later display. The augmented image 700 may be part of a video or series of images. When part of a video or series of images, features may be identified as they change (e.g., as an otoscope is moved). A base image may be captured by an otoscope and the augmented image 700 may be generated via object detection processing.

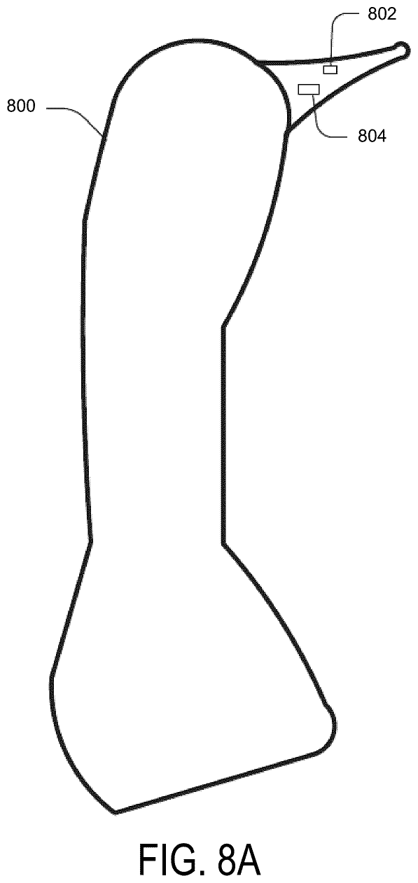

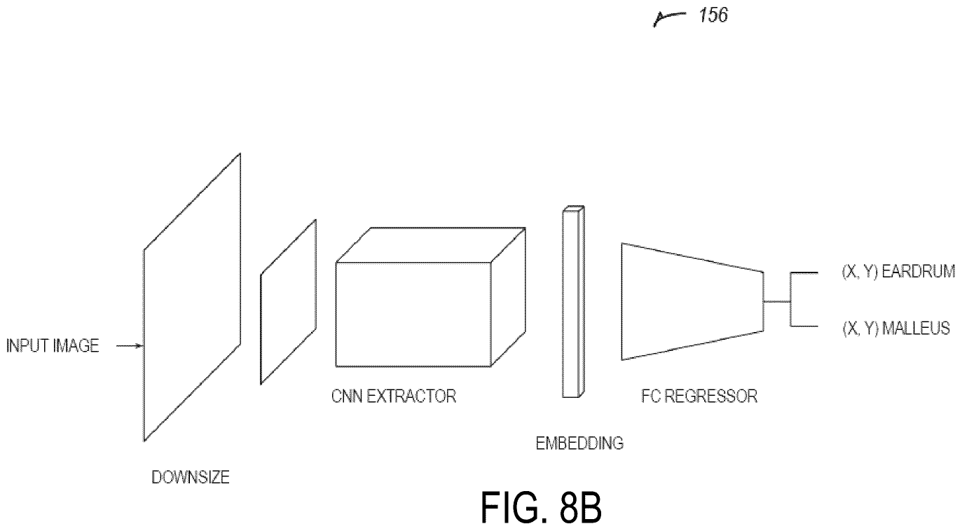

FIG. 8A illustrates an otoscope 800 with one or more additional components. For example, the otoscope 800 may include a thermometer 802. In an example, the otoscope 800 may include a sound-emitter 804. In some examples, the otoscope 800 may include both the thermometer 802 and the sound-emitter 804.

A digital infrared thermometer (e.g., thermometer 802) can be incorporated to the digital otoscope 800 with one or more LED illuminators (e.g., 6 LED illuminators). In an example, 2 of the illuminating LEDs can be replaced with IR sensors to detect basic ear temperatures. Given that the otoscope is already inserted in the ear to visualize ear canal and anatomies at the same time the thermometer 802 can measure heat emitted in the ear canal at the same time (or separately). A handheld device (e.g., the otoscope 800) can achieve dual-otoscope-thermometer with the same housing. In an example, a readout can be retrieved via Wi-Fi or Bluetooth connection to a mobile device.

Hearing screening can be achieved via a sound-emitting thermometer-otoscope-hearing screening device (e.g., the otoscope 800), for example in place of an earphone. In the otoscope 800, a beep of different volumes can be played via the otoscope 800. In an example, an on-screen questionnaire (e.g., on a mobile device) can query whether the person being examined can hear the sound. Based on the response, the examiner or person being examined can determine whether there is a reason to book an appointment with a hearing specialist. The 2-in-1 or 3-in-1 otoscope 800 combining a thermometer plus an otoscope, or an otoscope plus a hearing screening test, or all three, can be an effective tool to screen or monitor for otitis media or hearing loss at a low-resource setting, for example where access to all three devices is not possible.

FIG. 8B illustrates an example of an architecture for an object detection model. For example, such an architecture can be implemented in the object detection model 156 depicted in FIG. 1B. The object detection model can be configured to identify each of a malleus handle and an eardrum of a human patient. In an example, the object detection model can involve detecting spatial centroids of each of the malleus handle and the eardrum, such as including a possibility of adding more predictions (rotation, extent) downstream. In an example, the model can receive a scaled image (e.g., scaled to about 224×224 pixels) and the model can output a 4×1 vector, e.g., interpreted as the approximated or estimated X/Y coordinates of the malleus and eardrum, respectively. Such coordinates can be outputted in size-normalized form (e.g., (0, 1)) such as to maintain invariance against the image size used. In an example, the object detection model can include a convolutional neural network (CNN)-based feature extractor, followed by a fully connected regressor. In an example, the object detection model can be user-configurable (e.g., via a medical professional or a technician) via hyper-parameters, facilitating a relatively high degree of control in experimentation. In an example, a ResNet50-based featureextractors can be used, such as facilitating a classification backbone having a relatively high predictive power in low-dimensional regression settings. In an example, a 1000 feature output can be used, with ReLU activation through the regressor. In an example, the object detection model can be user-configurable via a network output activation parameter. Given the prediction can generally fall within a 0-1 normalized output range, inductive bias can indicate that a sigmoidal activation may represent the problem. In an example, the object detection model can also be user-configurable via tuning of hyperparameters such as learning rate (LR), batch size, momentum, weight decay, and anchor box sizes. In an example, Stochastic Gradient Descent (SGD) optimization can be used, with momentum also subject to tuning, and schedule based LR tied to the validation loss plateau. In an example, a 60/80/10 or similar split can be used.

In addition to the object detection model, a classification model can be included to determine a binary classification of whether an eardrum depicted in an image frame exhibits an indication of OM. Such a classification model can include or use a convolutional neural network (CNN). In an example, the binary classification model can include a DenseNet architecture, such as involving a favorable trade-off between accuracy and training time (e.g., in one example exhibiting an average accuracy of 95.59% for their 9-class dataset of 20,542 images). In an example, the DenseNet model can be pre-trained on a 1000 class ImageNet dataset. Such a pre-trained model can be altered such as to use two output classes in a final linear layer. Also, images can be downscaled toward a resolution of about 224×224 pixels to better be received by the DenseNet model. In an example, the weights of early layers in the model can be frozen while gradient descent can be performed to find optimal weights in the adjusted linear layer.

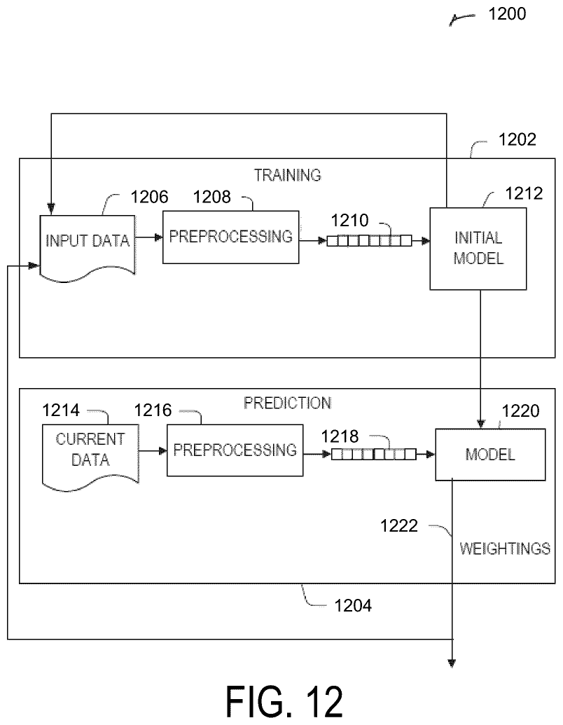

FIG. 12 illustrates machine learning engine for training and execution related to object detection, in accordance with at least one example of this disclosure. The machine learning engine may be deployed to execute at a mobile device (e.g., a cell phone) or a computer (e.g., a server). A system may calculate one or more weightings for criteria based upon one or more machine learning algorithms. FIG. 12 shows an example machine learning engine 1200 according to some examples of the present disclosure.

Machine learning engine 1200 uses a training engine 1202 and a prediction engine 1204. Training engine 1202 uses input data 1206, for example after undergoing preprocessing component 1208, to determine one or more features 1210. The one or more features 1210 may be used to generate an initial model 1212, which may be updated iteratively or with future labeled or unlabeled data (e.g., during reinforcement learning), for example to improve the performance of the prediction engine 1204 or the initial model 1212. An improved model may be redeployed for use.

The input data 1206 may include a set of images (e.g., frames of a video, a video, etc.), for example, of an ear, nose, or throat. The input data 1206 may include an image captured by an otoscope.

In the prediction engine 1204, current data 1214 (e.g., an image, a video frame, a video, etc. from an otoscope) may be input to preprocessing component 1216. In some examples, preprocessing component 1216 and preprocessing component 1208 are the same. The prediction engine 1204 produces feature vector 1218 from the preprocessed current data, which is input into the model 1220 to generate one or more criteria weightings 1222. The criteria weightings 1222 may be used to output a prediction, as discussed further below.

The training engine 1202 may operate in an offline manner to train the model 1220 (e.g., on a server). The prediction engine 1204 may be designed to operate in an online manner (e.g., in real-time, at a mobile device, at a physician's device at an otoscope, etc.). In some examples, the model 1220 may be periodically updated via additional training (e.g., via updated input data 1206 or based on labeled or unlabeled data output in the weightings 1222) or based on identified future data, such as by using reinforcement learning to personalize a general model (e.g., the initial model 1212) to a particular user, physician, type of otitis media, type of object to be detected, or the like.

Labels for the input data 1206 may include identification of an object in an image (e.g., a bounding box, a contour, a point label (e.g., a center or other point on an object), a feature, or the like).

The initial model 1212 may be updated using further input data 1206 until a satisfactory model 1220 is generated. The model 1220 generation may be stopped according to a specified criteria (e.g., after sufficient input data is used, such as 1,000, 10,000, 100,000 data points, etc.) or when data converges (e.g., similar inputs produce similar outputs).

The specific machine learning algorithm used for the training engine 1202 may be selected from among many different potential supervised or unsupervised machine learning algorithms. Examples of supervised learning algorithms include artificial neural networks, Bayesian networks, instance-based learning, support vector machines, decision trees (e.g., Iterative Dichotomiser 3, C9.5, Classification and Regression Tree (CART), Chi-squared Automatic Interaction Detector (CHAID), and the like), random forests, linear classifiers, quadratic classifiers, k-nearest neighbor, linear regression, Logistic Regression, and hidden Markov models. Examples of unsupervised learning algorithms include expectation-maximization algorithms, vector quantization, and information bottleneck method. Unsupervised models may not have a training engine 1202. In an example embodiment, a regression model is used and the model 1220 is a vector of coefficients corresponding to a learned importance for each of the features in the vector of features 1210, 1218. A reinforcement learning model may use Q-Learning, a deep Q network, a Monte Carlo technique including policy evaluation and policy improvement, a State-Action-Reward-State-Action (SARSA), a Deep Deterministic Policy Gradient (DDPG), or the like. An example model may include an AlexNet Model, an DenseNet Transfer Learning Model, a convolutional neural network (CNN), or the like.

Once trained, the model 1220 may output a vector (e.g., coordinates of a detected object), a bounding box, a center or other point on an object (e.g., an anchor point), information related to a detected object, or the like.

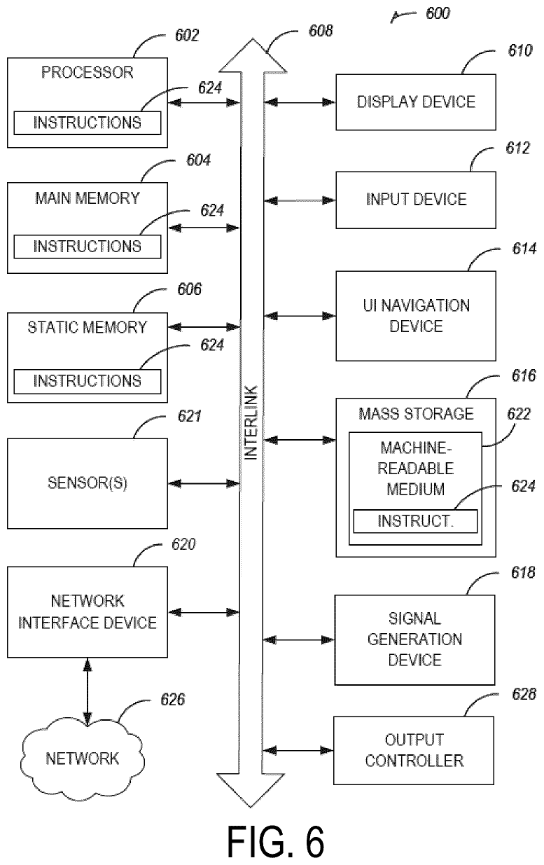

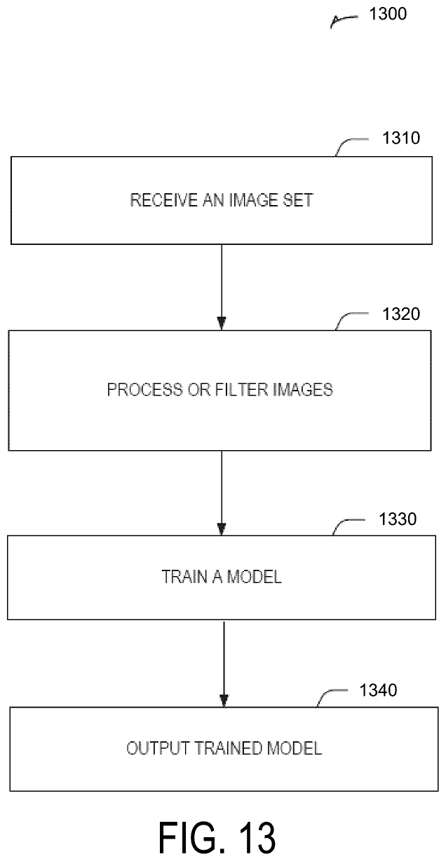

FIG. 13 illustrates a flowchart showing a technique 1300 for identifying an anatomical object of a middle ear in an image in accordance with at least one example of this disclosure. The technique 1300 may be implemented using one or more devices or systems described herein, such as the processor of FIG. 6, the platform 100 of FIG. 1A, the system 150 of FIG. 1B, etc.

The technique 1300 includes an operation 1310 to receive an image set, where each image corresponds to the ears of different human patients. For example, the image set can include images taken by a treating doctor or physician, such as during a surgical procedure or a diagnosis. In an example, images in the image set can be taken using an otoscope, e.g., via a camera attached thereto. In an example, each image in the image set can correspond with a diagnosis for the same patient.

The technique 1300 includes an operation 1320 to process or filter the images. The processing or filtering these images can be based on the identification of at least one target feature. For example, such target features may include a visible malleus handle, a visible eardrum, visible glare from a point light source, an image contrast level, a sharpness of an image, an image color, or other optical feature that is salient in the image. The processing or filtering may also involve augmenting the images to exhibit such target features more prominently, which can include techniques such as contrast correction, channel-wise color normalization, spatial distortion, saturation adjustment, intensity adjustment, labeling, etc.

The technique 1300 includes an operation 1330 to train a model, e.g., using aspects of the processed or filtered images in a data set as training data. The training process can adapt the model to recognize and determine the presence of the anatomical object, such as a malleus, an eardrum, or both. In an example, the model is a machine learning model such as a convolutional neural network (CNN), which is particularly suited for image recognition tasks due to its ability to preserve the spatial hierarchy in images.

The technique 1300 includes an operation 1340 to output the trained model for practical use. For example, the outputted model can be used to determine whether an image frame captured by an otoscope includes the anatomical object and relevant information necessary for the diagnosis of Otitis Media. The trained model can effectively utilize the detailed features recognized and learned during the training phase to provide accurate and reliable diagnostic outputs.

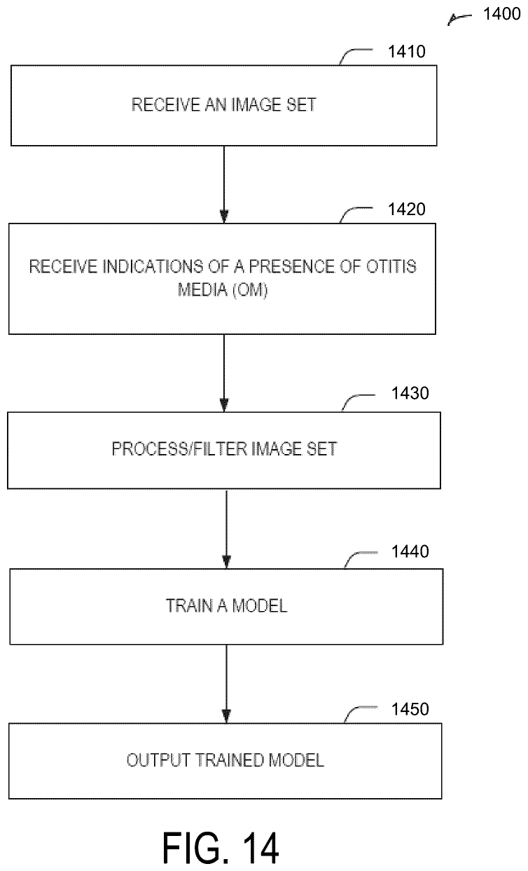

FIG. 14 illustrates a flowchart showing a technique 1400 for identifying a presence of Otitis Media (OM), e.g., at or near a middle ear in an image frame in accordance with at least one example of this disclosure. The technique 1400 may be implemented using one or more devices or systems described herein, such as the processor of FIG. 6, the platform 100 of FIG. 1A, the system 150 of FIG. 1B, etc.

The technique 1400 includes an operation 1410 to receive an image set, where each image corresponds with a middle car of a plurality of different human patients. Such images can define biophysical features of the respective patients.

The technique 1400 includes an operation 1420 to receive respective indications of the presence of Otitis Media (OM) corresponding to the images and the patients are received. For example, the respective indications of a presence of OM can include respective symptoms or respective previous diagnoses of the patients. Such data can be used to label or otherwise analyze the image set. For example, receiving corresponding indications of a presence of OM can aid in a downstream supervised learning process, enabling the model to learn from diagnostic outcomes.

The technique 1400 includes an operation 1430 to process or filter the image set to include or retain only images in the set that exhibit at least one target feature. For example, the at least one target feature can include a visible malleus handle or a visible eardrum. The at least one target feature can be an identified lighting property of the middle car of a patient, such as a lack of glare, a threshold amount of image contrast, or an overall level of lightness in the middle ear to more effectively detect the presence of certain features in the middle car. The processing or filtering may also involve augmenting the images to enhance these features more prominently, which could include techniques such as contrast correction, channel-wise color normalization, spatial distortion, or labeling. Such augmentations can aid in emphasizing the features critical for diagnosing OM, such as enhancing them to improve detection e.g., via a machine learning model.

The technique 1400 includes an operation 1440 to training a model via the processed or filtered images and the corresponding indications of OM. For example, the model can use the training data to identify biophysical features corresponding with OM in an image frame captured by an otoscope. The model, such as a convolutional neural network (CNN), can be trained to correlate specific visual patterns with the presence of OM. The training process can be further aided by the inclusion of specific biophysical features indicative of various types of Otitis Media, such as Acute Otitis Media (AOM), Chronic Suppurative Otitis Media (CSOM), or Serous Otitis Media (OME) in the training data.

The technique 1400 includes an operation 1450 to output the trained model for clinical use in car nose and throat disease state diagnostic support. For example, the model can determine whether an indication of Otitis Media is present in the image frame. The model can also provide diagnostic information to assists a healthcare professional in decision making regarding treatment and management of OM.

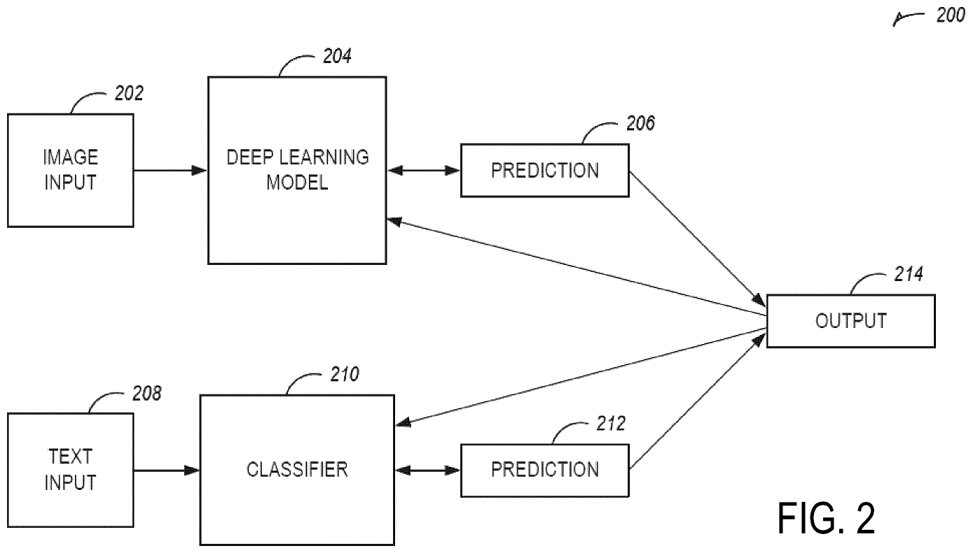

FIG. 2 illustrates a block diagram 200 for training and implementing a model and classifier for predicting outcomes related to car nose and throat disease state in accordance with at least one example of this disclosure. The block diagram 200 includes a deep learning model 204 and a classifier 210, which each receive inputs and output a prediction. The deep learning model 204 receives an image input 202 and outputs an image-based prediction 206 and the classifier 210 receives a text input 208 and outputs a text-based prediction 212. The image-based prediction 206 and the text-based prediction 212 may be combined as an output 214. The output may include either prediction 206 or 212, or a combination, such as by multiplying likelihoods together, taking an average likelihood, using one of the results as a threshold, or the like.

In some examples, the prediction 206 may be fed back to the deep learning model 204, for example as a label for the image input 202 when training the deep learning model 204. In another example, the prediction 206 may be fed back to the deep learning model 204 as a side input (e.g., for use in a recurrent neural network). The output 214 may be used similarly to the prediction 206 for feedback or continuous retraining the model for the purpose of obtaining the robust model. The prediction 212 may be used similarly with the classifier 210.

In an example, images may be represented as a 2D grid of pixels. CNNs as the deep learning network may be used for data with a grid-like structure.

Applying CNNs in the case of diagnosis, a medical image may be analyzed for a binary classification or a probability problem, giving the ill versus normal and the likelihood of illness as a reference for a doctor's decision.

Image only techniques may be improved with new architecture with additional layers, more granular features on the images, and optimized weights in custom model specific for AOM that may be deployed over mobile computing.

In one embodiment, Tensorflow (of Google) architecture may be used for the deep learning-based image classification (e.g., for implementing the deep learning model 204). A set of proprietary architecture components including selected model type, loss function, batch size, and a threshold may be used as input for classification predictions. The selection criteria of the architecture components may include optimal performance in recall and precision and real number metric values were easy to translate and manipulate for mixing with text classification.

Testing on the validation dataset may include an F1 value of 72% for image classification in which F1 value is defined as a tradeoff between precision and recall.

A multi-model model using a TensorFlow model may be used to achieve more accurate results. In one embodiment, the multi-model classification (e.g., at the classifier 210) combines image and text classification, mixing their confidence values and generate a new decision based on threshold.

In the above mentioned multi-model classification embodiment (e.g., classifier 210), a grid search method may be used with two parameters for improved performance including a weight of image and text results (e.g., how much is the image used and text, respectively), or setting of a threshold for making a binary classification. For example, when the combined confidence value, such as probability is 0.7, setting threshold to 0.6 and 0.8 may yield opposite decisions.

In an example, one challenge includes using short text classification with very limited context. In an example, users may choose from a given set of symptoms. Although they are short texts, the vocabulary may be confined to a small set, for example considering some symptoms that are specifically for a particular illness. That is, if for all or most of the ill cases, some symptoms exist in the training dataset, and exclusively not in the normal case data. The classifier to make these symptoms may include strong indicators of the illness for drawing conclusions with high confidence values.

In an example, a support vector machine (SVM) may be used as a text classification algorithm for the classifier 210. In some examples, the SVM may have a difficult output to interpret or combine with the result from the image model. A Logistic Regression or Naives Bayes classifier may be chosen as a tool for text classification because it is easy to implement and interpret. This may help better design the symptom descriptions.

The AI diagnosis assistant system uses the deep learning model 204, for example with a convolutional neural network (CNN).

In an example, an image classification may be performed on the input image 202 using the deep learning model. An object detection technique may be used on the input image, for example before it is input to the deep learning model. The object detection may be used to determine whether the image properly captured an eardrum or a particular area of an eardrum. For example, the object detection may detect a Malleus Handle on a captured eardrum. After detecting the Malleus Handle, the image may be segmented (e.g., with two perpendicular lines to create four quadrants). The segmented portions of the image may be separately classified with the deep learning model as input images in some examples.

Another object detection may be used, together (before, after, or concurrently) or separately from the above object detection. This object detection may include detecting whether a round or ellipse shaped eardrum appears in a captured image. This object detection may include determining whether the round or ellipse shaped eardrum occupies at least a threshold percentage (e.g., 50%, 75%, 90%, etc.) of the captured image. In some examples, clarity or focus of the image (e.g., of the round or ellipse shaped eardrum portion of the image) may be checked during object detection.

In some examples, a set of deep learning trained models may be used to create more robust and reliable model for real-world applications. For example, a different model may be used for each segmented portion of an image. In an example where a captured image is segmented into four quadrants based on object detection of a Malleus Handle, four models may be trained or used. An output of a deep learning model may include a number, such as a real number between 0 and 1. When using more than one model, a prediction indication may be generated based on values from a set of or all of the models used. For example, an average, medium, or other combination of model output numbers may be used to form a prediction. The prediction may indicate a percentage likelihood of a disease state of a patient (e.g., an car infection in an car or a portion of an car).

Data may be collected for training the deep learning model 204 from consenting patients, in some examples. An image may be captured of a patient, such as by a clinician (e.g., a doctor), by the patient, by a caretaker of the patient, or the like. The image may be labeled with an outcome, such as a diagnosis from a doctor (e.g., that an ear infection was present). In some examples, other data may be collected from the patient, such as symptoms. The other data may be used as an input to the classifier 210. An output of the classifier (e.g., prediction 212) may be used to augment the output of the deep learning model, as discussed further below. The other data may be selected by a patient, caretaker, or clinician, such as by text input, text drop down selection on a user interface, spoken audio to text capture, or the like. The image and text data may be captured together (e.g., during a same session using an application user interface) or separately (e.g., text at an intake phase, and an image at a diagnostic phase).

A system may be trained using a multi-modal approach, including image and text classification. For an image model (e.g., deep learning model 204), one or more CNNs may be used, for example. For the classifier 210, in an example, a support vector machine classifier, Naïve Bayes algorithm, or other text classifier may be used. The deep learning model 204 and the classifier 210 may output separate results (e.g., predictions 206 and 212 of likelihood of the presence of a disease state, such as an ear infection). The separate results may be combined, such as by multiplying percentage predictions, using an average, using one as a confirmation or threshold for the other (e.g., not using the text output if the image input is below a threshold), or the like as the output 214.

During an inference use of the deep learning model 204 and the classifier 210, a user may receive a real time or near real time prediction of a disease state for use in diagnosis. The inference may be provided to a user locally or remotely. In the local example, a doctor may capture an image of a patient, and text may be input by the patient or the doctor. The doctor may then view the prediction, which may be used to diagnose the patient. In the remote example, the patient may capture the image and input the text, which may be used to generate the inference. In the remote example, the inference may be performed at a patient device, at a doctor operated device, or remote to both the doctor and the patient (e.g., at a server). The results may be output for display on the doctor operated device (e.g., a phone, a tablet, a dedicated diagnosis device, or the like). The doctor may then communicate a diagnosis to the patient, such as via input in an application which may be sent to the patient, via a text message, via a phone call, via email, etc. In the remote example, a doctor may view a patient camera (e.g., an otoscope) live. In an example, the doctor may cause capture of an image at the doctor's discretion. In another example, the patient may record video, which the doctor may use to capture an image at a later time.

In a real-time consult example, a user may stream video to a doctor and the doctor may take a snapshot image. The doctor may receive an input symptom description from the patient. A UI component (for example, a button) may be used to allow the doctor to query the model to perform a prediction for the possibility of an ear infection or other disease state. In another example, the user may capture an image or input symptoms before the doctor consultant and send the information to the doctor. The doctor may import the data to the model or ask for the prediction.

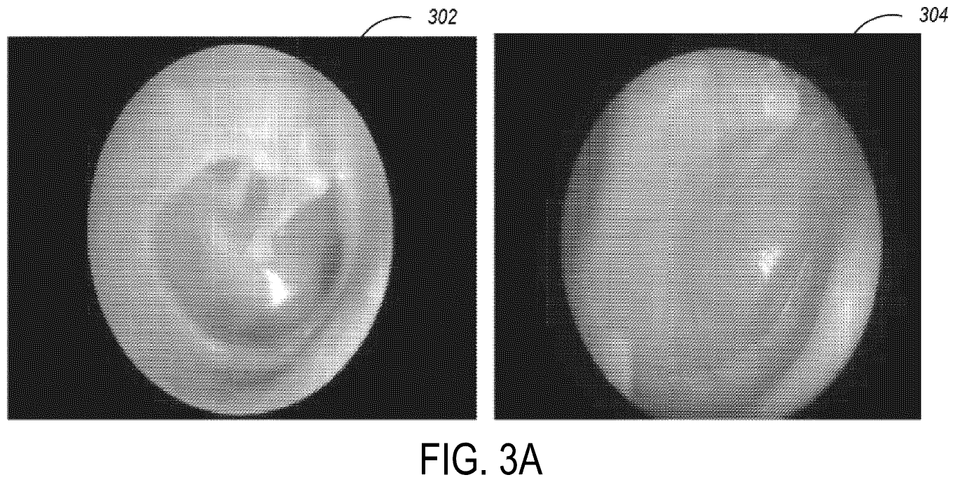

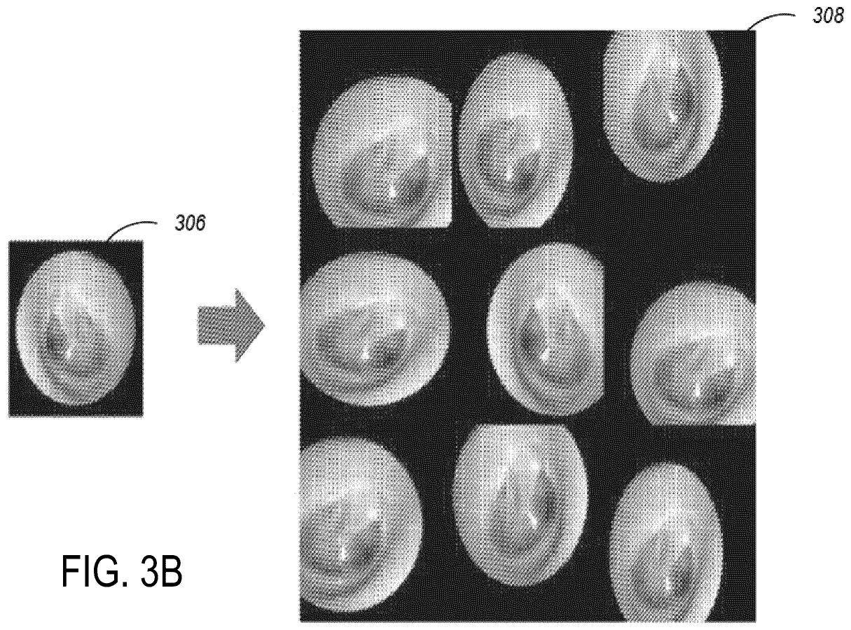

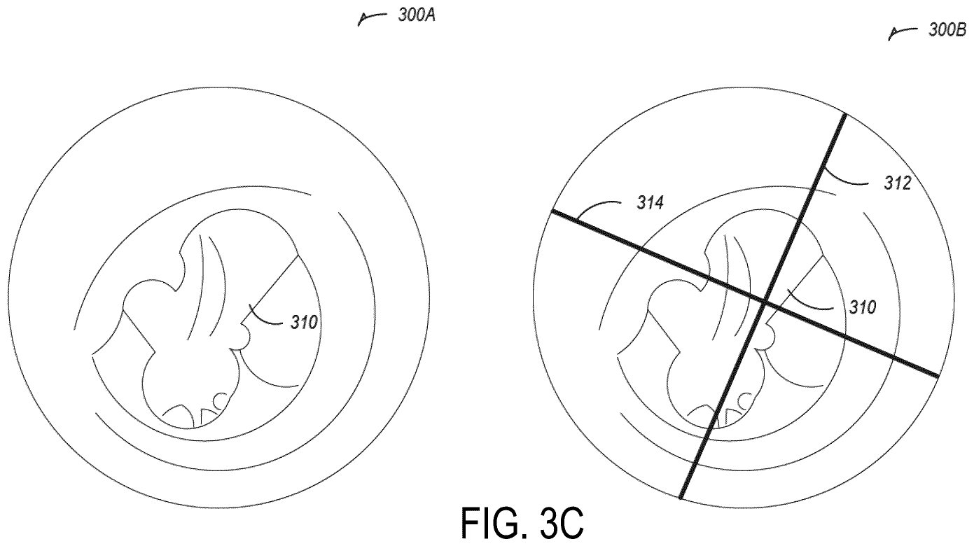

FIG. 3A illustrates examples of a healthy eardrum and an infected eardrum in accordance with at least one example of this disclosure.

A healthy eardrum appears clear and pinkish-gray, whereas an infected one will appear red and swollen due to fluid buildup behind the membrane.

FIG. 3B illustrates an example of data augmentation to generate training data in accordance with at least one example of this disclosure.

Data augmentation may be used to create a larger dataset for training the algorithm. In one embodiment, a combination of several data augmentation approaches is adopted, including translation, rotation and scaling. Additional augmentation methods, said color and brightness adjustments, are introduced if needed.

In one embodiment, by using python library of Keras (https://keras.io/), an original image can generate 10 images through rotating, flipping, contrast stretching, histogram equalization, etc. The new images still retain the underlying patterns among pixels and serve as random noises to help train the classifier.

An eardrum may be visualized in several orientations and by augmenting the training data with rotated examples the algorithm will be robust to changes in rotation.

The actual size of eardrum changes as a patient grows and varies from patient to patient, additionally, the size of the eardrum in an otoscope image will vary depending on the position of the device in the ear. The image dataset may be augmented by using scaling to make the algorithm robust to images of varying size.

FIG. 3C illustrates an example of image segmentation in accordance with at least one example of this disclosure. The image segmentation may include an object detection, which is shown in a first image 300A of an ear of a patient. The object detection may be used to identify a Malleus Handle or other anatomical feature at location 310 of an ear drum of the car of the patient. After identification of the Malleus Handle or other anatomical feature, the image may be segmented, for example into quadrants. The quadrants may be separated according to a line 312 (which may not actually be drawn, but is shown for illustrative purposes in a second image 300B of FIG. 3C) that bisects, is parallel to, or otherwise references the Malleus Handle or other anatomical feature. A second line 314 (again, shown in the second image 300B of FIG. 3C, but not necessarily drawn on the image in practice) may be used to further segment the image into the quadrants by bisecting the line 312, for example, or otherwise intersecting with the line 312. Further segmentation may be used (e.g., additional lines offset from the lines 312 or 314) in some examples. Each portion of the segmented image in 300B may be used with a model (e.g., a separate model or a unified model) for detecting disease state as described herein.

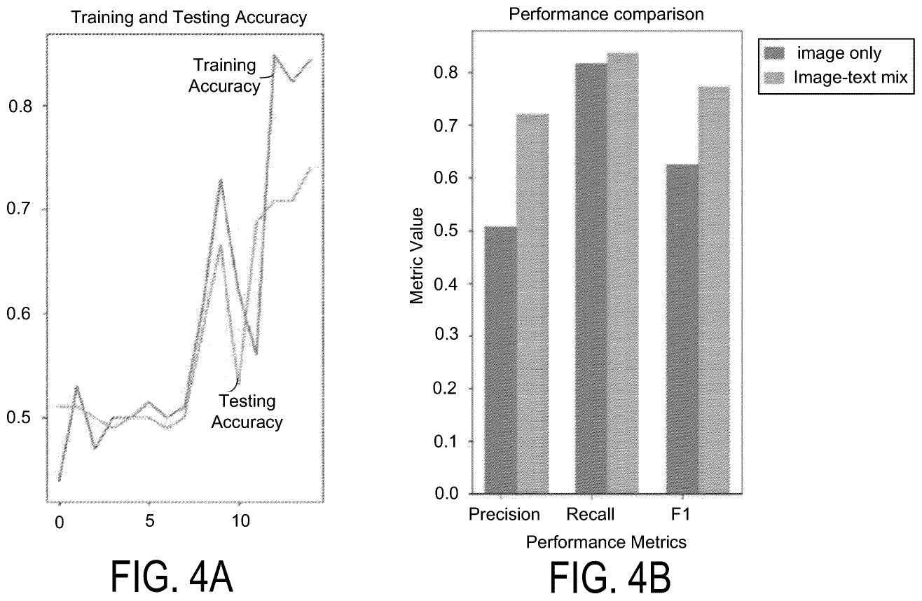

FIGS. 4A-4B illustrate results of image and text classification predictions in accordance with at least one example of this disclosure.

The classification of eardrum images is complicated. Off-the-shelf models, such as AWS Rekognition (of Amazon) and Azure Custom Vision (of Microsoft) may be used for testing. Both services yield high accuracy.

As FIG. 4A shows, the model (e.g., combining image and text classification) training tends to converge well with a low training loss and evaluation accuracy reaches above 70%.

Testing on the validation dataset yields an F1 value of 72% for image classification in which F1 value is defined as a tradeoff between precision and recall. As shown in FIG. 4B, the multi-model classification brings up the overall accuracy from original 72%) to over 90%. This proves the effectiveness of the multi-model classification method.

Once the device collects sufficient data, the data may be used to train selected off-the-shelf models and further develop the custom model.

In order to select one off-the-shelf model that provides the best Positive Predicated Value (Precision) and Sensitivity (Recall), 500 normal and 300-500 AOM images are tested in off-the-shelf models to compare and contrast performance. In some embodiments, off-the-shelf models adopted include Alexnet, DenseNet, GoogLeNet, ResNet, Inception-V3, SqueezeNet, MobileNet-V2, public packages Microsoft Custom Vision, or Amazon Rekognition.

Transfer learning may be used to build the custom architecture. In one embodiment, 500 validation images with blinded labels are used to test the algorithm for at least 90% PPV and 95% sensitivity in identifying an AOM. An iterative approach may be taken once additional training images become available to optimize the algorithm.

In one embodiment, the algorithm may be built-in to an app used for clinical validation to classify, for example, at least 50 new test images, blinded against clinical diagnosis by a provider. A usability interview may be conducted to collect feedback from the provider regarding User Experience Design and result interpretation of the model output for future improvement.

In some embodiments, the algorithm may be used to support diagnosis of other ear, nose, and throat ailments for adults and children. In performing expansion of the classification to identify images not classified as normal or AOM, including but not limited to Obstructing Wax or Foreign Bodies (O/W), Otitis Media with Effusion (OME), or Chronic Suppurative Otitis Media with Perforation (CSOM with Perforation). Image augmentation may increase the training data size. A similar iterative process may be performed, characterized, compared, or optimized as that for AOM.

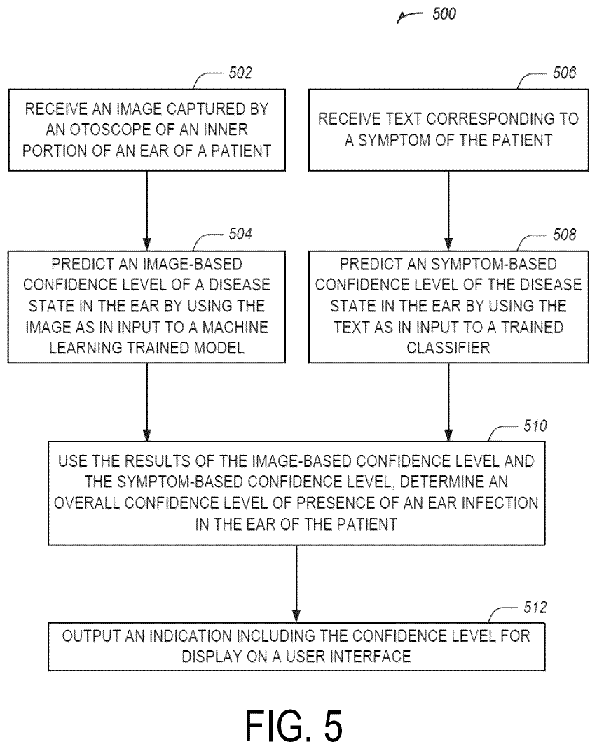

FIG. 5 illustrates a flowchart showing a technique 500 for generating an car disease state prediction to assist diagnosis of an ear disease in accordance with at least one example of this disclosure. The technique 500 may be performed by a processor by executing instructions stored in memory.

The technique 500 includes an operation 502 to receive an image captured by an otoscope of an inner portion of an car of a patient. The technique 500 includes an operation 504 to predict an image-based confidence level of a disease state in the car by using the image as in input to a machine learning trained model. The machine learning trained model may include a convolutional neural network model.

The technique 500 includes an operation 506 to receive text corresponding to a symptom of the patient. In an example, receiving the text may include receiving a selection from a list of symptoms. In another example, receiving the text may include receiving user input custom text.

The technique 500 includes an operation 508 to predict a symptom-based confidence level of the disease state in the car by using the text as in input to a trained classifier. The trained classifier may include a support vector machine (SVM) classifier or a Logistic Regression model classifier.

The technique 500 includes an operation 510 to use the results of the image-based confidence level and the symptom-based confidence level to determine an overall confidence level of presence of an ear infection in the car of the patient. In an example, the overall confidence level may include a confidence level output from the machine learning trained model multiplied by a confidence level output from the trained classifier. In other examples, the overall confidence level may include an average of the confidence level output from the machine learning trained model and the confidence level output from the trained classifier. In some examples, the overall confidence level may use one of the confidence level output from the machine learning trained model and the confidence level output from the trained classifier as a threshold, and output the other. The technique 500 includes an operation 512 to output an indication including the confidence level for display on a user interface.

The technique 500 may include segmenting the image, and wherein the input to the machine learning trained model includes each segmented portion of the image. In this example, the technique 500 may include performing object detection on the image to identify a Malleus Handle in the image, and wherein segmenting the image includes using the identified Malleus Handle as an axis for segmentation. In an example, the technique 500 may include performing object detection on the image to identify whether the image captures an entirety of an ear drum of the car.

FIG. 6 illustrates a block diagram of an example machine 600 upon which any one or more of the techniques discussed herein may perform in accordance with some embodiments. In alternative embodiments, the machine 600 may operate as a standalone device and/or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610, an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensor. The machine 600 may include an output controller 628, such as a serial (e.g., Universal Serial Bus (USB), parallel, or other wired or wireless (e.g., infrared (IR), near field communication (NFC), etc.) connection to communicate and/or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine readable media.

While the machine readable medium 622 is illustrated as a single medium, the term “machine readable medium” may include a single medium or multiple media (e.g., a centralized or distributed database, and/or associated caches and servers) configured to store the one or more instructions 624. The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

Each of the following non-limiting examples may stand on its own, or may be combined in various permutations or combinations with one or more of the other examples.

Example 1 is a method for generating an car disease state prediction to assist diagnosis of an car disease, the method comprising: receiving, at one or more processors, an image captured by an otoscope of an inner portion of an car of a patient; identifying, at the one or more processors, an anatomical object of the car from the image by using a trained object detection model, wherein identifying the anatomical object comprises: augmenting, by an augmentation engine and based at least in part on the image captured by the otoscope, the image captured by the otoscope to generate an augmented image; identifying, by a bounding engine and based at least in part on the image captured by the otoscope, one or more regions of interest of the image captured by the otoscope; retraining, at the one or more processors, the trained object detection model based on the one or more regions of interest and the augmented image; and identifying, by the trained object detection model, the anatomical object; in response to identifying the anatomical object, predicting, at the one or more processors, an image-based confidence level of a disease state in the car by using a plurality of machine learning trained models, wherein predicting the image-based confidence level comprises: segmenting the image captured by the otoscope or the augmented image into a plurality of segments; predicting a plurality of values using a plurality of machine learning models, wherein each of the plurality of machine learning models takes a respective segment of the plurality of segments as input; and generating the image-based confidence level based on the plurality of values; receiving text corresponding to a symptom of the patient; predicting a symptom-based confidence level of the disease state in the ear by using the text as in input to a classifier; combining the image-based confidence level and the symptom-based confidence level to generate an overall confidence level of presence of an ear infection in the car of the patient; and outputting an indication including the confidence level for display on a user interface.

In Example 2, the subject matter of Example 1 includes, wherein augmenting the image captured by the otoscope comprises at least one of contrast correction, channel-wise color normalization, spatial distortion, or labeling.

In Example 3, the subject matter of Example 2 includes, wherein the contrast correction comprises interpolation between a darkest pixel and a lightest pixel of the image, and wherein spatial distortion comprises at least two of cropping, scaling, and rotation of the image.

In Example 4, the subject matter of Examples 1-3 includes, wherein the anatomical object comprises a Malleus Handle of a human ear, and wherein segmenting the image includes using the identified Malleus Handle as an axis for segmentation, and wherein each machine learning model of the plurality of machine learning models is trained to predict the image-based confidence level for a respective segment of the plurality of segments based on the identified Malleus Handle.

In Example 5, wherein identifying the anatomical object of the car by using the trained object detection model further comprising: identifying, at the one or more processors, an entirety of an ear drum of the ear, wherein the image is augmented in response to identifying the entirety of the ear drum of the ear.

In Example 6, the subject matter of Examples 1-5 includes, wherein determining the overall confidence level includes multiplying the image-based confidence level output from the plurality of machine learning models by the symptom-based confidence level output from the classifier.

In Example 7, the subject matter of Examples 1-6 includes, wherein receiving the text including receiving a selection from a list of symptoms.

In Example 8, the subject matter of Examples 1-7 includes, wherein the classifier is a support vector machine (SVM) classifier, a Logistic Regression model classifier, or Naives Bayes classifier.

In Example 9, the subject matter of Examples 1-8 includes, wherein the machine learning trained model is a convolutional neural network model.

Example 10 is a system for generating an car disease state prediction to assist diagnosis of an car disease, the system comprising: processing circuitry; and memory including instructions, which when executed, cause the processing circuitry to: receive an image captured by an otoscope of an inner portion of an car of a patient; process the image to detect an anatomical object by: augmenting the image captured by the otoscope to generate an augmented image; identifying one or more regions of interest of the image captured by the otoscope; retraining the trained object detection model based on the one or more regions of interest and the augmented image; and detecting the anatomical object by the trained object detection model; segment the image captured by the otoscope or the augmented image into a plurality of segments; predict a plurality of values using the plurality of machine learning models, wherein each of the plurality of machine learning models takes a respective segment of the plurality of segments as input; and generate an image-based confidence level based on the plurality of values; receive symptom information corresponding to the patient; predict a symptom-based confidence level using a classifier based on the symptom information; combine the image-based confidence level and the symptom-based confidence level to generate an overall disease state prediction for the patient; and output an indication of the overall disease state prediction for display on a user interface.

In Example 11, the subject matter of Example 10 includes, wherein augmenting the image captured by the otoscope comprises at least one of contrast correction, channel-wise color normalization, spatial distortion, or labeling.

In Example 12, the subject matter of Example 11 includes, wherein the contrast correction comprises interpolation between a darkest pixel and a lightest pixel of the image, and wherein spatial distortion comprises at least two of cropping, scaling, and rotation of the image.

In Example 13, the subject matter of Examples 10-12 includes, wherein the anatomical object comprises a Malleus Handle of a human ear, and wherein segmenting the image includes using the identified Malleus Handle as an axis for segmentation, and wherein each machine learning model of the plurality of machine learning models is trained to predict the image-based confidence level for a respective segment of the plurality of segments based on the identified Malleus Handle.

In Example 14, the subject matter of Examples 10-13 includes, wherein identifying the anatomical object of the ear by using the trained object detection model further comprising: identifying, at the one or more processors, an entirety of an ear drum of the ear, wherein the image is augmented in response to identifying the entirety of the ear drum of the ear.

In Example 15, the subject matter of Examples 10-14 includes, wherein the instructions further cause the processing circuitry to multiply the image-based confidence level output from the plurality of machine learning models by the symptom-based confidence level output from the classifier.

In Example 16, the subject matter of Examples 10-15 includes, wherein to receive the text, the instructions further cause the processing circuitry to receive a selection from a list of symptoms.

In Example 17, the subject matter of Examples 10-16 includes, wherein the classifier is a support vector machine (SVM) classifier, a Logistic Regression model classifier, or a Naives Bayes classifier.

In Example 18, the subject matter of Examples 10-17 includes, wherein the machine learning trained model is a convolutional neural network model.

Example 19 is at least one machine-readable medium including instructions for generating an ear disease state prediction to assist diagnosis of an ear disease, which when executed by processing circuitry, cause the processing circuitry to perform operations to: receive, at one or more processors, an image captured by an otoscope of an inner portion of an ear of a patient; identify, at the one or more processors, an anatomical object of the ear from the image by using a trained object detection model, wherein identifying the anatomical object comprises: augment, by an augmentation engine and based at least in part on the image captured by the otoscope, the image captured by the otoscope to generate an augmented image; identify, by a bounding engine and based at least in part on the image captured by the otoscope, one or more regions of interest of the image captured by the otoscope; retrain, at the one or more processors, the trained object detection model based on the one or more regions of interest and the augmented image; and identify, by the trained object detection model, the anatomical object; in response to identifying the anatomical object, predict, at the one or more processors, an image-based confidence level of a disease state in the ear by using a plurality of machine learning models, wherein predicting the image-based confidence level comprises: segment the image captured by the otoscope or the augmented image into a plurality of segments; predict a plurality of values using a plurality of machine learning models, wherein each of the plurality of machine learning models takes a respective segment of the plurality of segments as input; and generate the image-based confidence level based on the plurality of values; receive text corresponding to a symptom of the patient; predict a symptom-based confidence level of the disease state in the ear by using the text as in input to a classifier; combine the image-based confidence level and the symptom-based confidence level to generate an overall confidence level of presence of an ear infection in the ear of the patient; and output an indication including the confidence level for display on a user interface.

In Example 20, the subject matter of Example 19 includes, wherein the classifier is a support vector machine (SVM) classifier, a Logistic Regression model classifier, or Naives Bayes classifier and wherein the machine learning trained model is a convolutional neural network model.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

What is claimed is:

1. A method for generating an ear disease state prediction to assist diagnosis of an ear disease, the method comprising:
receiving, at one or more processors, an image captured by an otoscope of an inner portion of an ear of a patient;
identifying, at the one or more processors, an anatomical object of the ear from the image by using a trained object detection model, wherein identifying the anatomical object comprises:
augmenting, by an augmentation engine and based at least in part on the image captured by the otoscope, the image captured by the otoscope to generate an augmented image;
identifying, by a bounding engine and based at least in part on the image captured by the otoscope, one or more regions of interest of the image captured by the otoscope;
retraining, at the one or more processors, the trained object detection model based on the one or more regions of interest and the augmented image; and
identifying, by the trained object detection model, the anatomical object;
in response to identifying the anatomical object, predicting, at the one or more processors, an image-based confidence level of a disease state in the ear by using a plurality of machine learning trained models, wherein predicting the image-based confidence level comprises:
segmenting the image captured by the otoscope or the augmented image into a plurality of segments;
predicting a plurality of values using a plurality of machine learning models, wherein each of the plurality of machine learning models takes a respective segment of the plurality of segments as input; and
generating the image-based confidence level based on the plurality of values;
receiving text corresponding to a symptom of the patient;
predicting a symptom-based confidence level of the disease state in the ear by using the text as in input to a classifier;
combining the image-based confidence level and the symptom-based confidence level to generate an overall confidence level of presence of an ear infection in the ear of the patient; and
outputting an indication including the confidence level for display on a user interface.

2. The method of claim 1, wherein augmenting the image captured by the otoscope comprises at least one of contrast correction, channel-wise color normalization, spatial distortion, or labeling.

3. The method of claim 2, wherein the contrast correction comprises interpolation between a darkest pixel and a lightest pixel of the image, and wherein spatial distortion comprises at least two of cropping, scaling, and rotation of the image.

4. The method of claim 1, wherein the anatomical object comprises a Malleus Handle of a human ear, and wherein segmenting the image includes using the identified Malleus Handle as an axis for segmentation, and wherein each machine learning model of the plurality of machine learning models is trained to predict the image-based confidence level for a respective segment of the plurality of segments based on the identified Malleus Handle.

5. The method of claim 1, wherein identifying the anatomical object of the ear by using the trained object detection model further comprising:
identifying, at the one or more processors, an entirety of an ear drum of the ear, wherein the image is augmented in response to identifying the entirety of the ear drum of the ear.

6. The method of claim 1, wherein determining the overall confidence level includes multiplying the image-based confidence level output from the plurality of machine learning models by the symptom-based confidence level output from the classifier.

7. The method of claim 1, wherein receiving the text including receiving a selection from a list of symptoms.

8. The method of claim 1, wherein the classifier is a support vector machine (SVM) classifier, a Logistic Regression model classifier, or Naives Bayes classifier.

9. The method of claim 1, wherein the machine learning trained model is a convolutional neural network model.

10. A system for generating an ear disease state prediction to assist diagnosis of an ear disease, the system comprising:
a processing circuitry; and
a memory including instructions, which when executed, cause the processing circuitry to:
receive an image captured by an otoscope of an inner portion of an ear of a patient;
process the image to detect an anatomical object by:
augmenting the image captured by the otoscope to generate an augmented image;
identifying one or more regions of interest of the image captured by the otoscope;
retraining the trained object detection model based on the one or more regions of interest and the augmented image; and
detecting the anatomical object by the trained object detection model;
segment the image captured by the otoscope or the augmented image into a plurality of segments;
predict a plurality of values using the plurality of machine learning models, wherein each of the plurality of machine learning models takes a respective segment of the plurality of segments as input;
generate an image-based confidence level based on the plurality of values;
receive symptom information corresponding to the patient;
predict a symptom-based confidence level using a classifier based on the symptom information;
combine the image-based confidence level and the symptom-based confidence level to generate an overall disease state prediction for the patient; and
output an indication of the overall disease state prediction for display on a user interface.

11. The system of claim 10, wherein augmenting the image captured by the otoscope comprises at least one of contrast correction, channel-wise color normalization, spatial distortion, or labeling.

12. The system of claim 11, wherein the contrast correction comprises interpolation between a darkest pixel and a lightest pixel of the image, and wherein spatial distortion comprises at least two of cropping, scaling, and rotation of the image.

13. The system of claim 10, wherein the anatomical object comprises a Malleus Handle of a human ear, and wherein segmenting the image includes using the identified Malleus Handle as an axis for segmentation, and wherein each machine learning model of the plurality of machine learning models is trained to predict the image-based confidence level for a respective segment of the plurality of segments based on the identified Malleus Handle.

14. The system of claim 10, wherein identifying the anatomical object of the ear by using the trained object detection model further comprising:
identifying, at the one or more processors, an entirety of an ear drum of the ear, wherein the image is augmented in response to identifying the entirety of the ear drum of the ear

15. The system of claim 10, wherein the instructions further cause the processing circuitry to multiply the image-based confidence level output from the plurality of machine learning models by the symptom-based confidence level output from the classifier.

16. The system of claim 10, wherein to receive the text, the instructions further cause the processing circuitry to receive a selection from a list of symptoms.

17. The system of claim 10, wherein the classifier is a support vector machine (SVM) classifier, a Logistic Regression model classifier, or Naives Bayes classifier.

18. The system of claim 10, wherein the machine learning trained model is a convolutional neural network model.

19. At least one machine-readable medium including instructions for generating an ear disease state prediction to assist diagnosis of an ear disease, which when executed by processing circuitry, cause the processing circuitry to perform operations to:
receive, at one or more processors, an image captured by an otoscope of an inner portion of an ear of a patient;
identify, at the one or more processors, an anatomical object of the ear from the image by using a trained object detection model, wherein identifying the anatomical object comprises:
augment, by an augmentation engine and based at least in part on the image captured by the otoscope, the image captured by the otoscope to generate an augmented image;
identify, by a bounding engine and based at least in part on the image captured by the otoscope, one or more regions of interest of the image captured by the otoscope;
retrain, at the one or more processors, the trained object detection model based on the one or more regions of interest and the augmented image; and
identify, by the trained object detection model, the anatomical object;
in response to identifying the anatomical object, predict, at the one or more processors, an image-based confidence level of a disease state in the ear by using a plurality of machine learning models, wherein predicting the image-based confidence level comprises:
segment the image captured by the otoscope or the augmented image into a plurality of segments;
predict a plurality of values using a plurality of machine learning models, wherein each of the plurality of machine learning models takes a respective segment of the plurality of segments as input; and
generate the image-based confidence level based on the plurality of values;
receive text corresponding to a symptom of the patient;
predict a symptom-based confidence level of the disease state in the ear by using the text as in input to a classifier;
combine the image-based confidence level and the symptom-based confidence level to generate an overall confidence level of presence of an ear infection in the ear of the patient; and
output an indication including the confidence level for display on a user interface.

20. The at least one machine-readable medium of claim 19, wherein the classifier is a support vector machine (SVM) classifier, a Logistic Regression model classifier, or Naives Bayes classifier and wherein the machine learning trained model is a convolutional neural network model.
